(19) United States
(12) Patent Application Publication
(10) Pub. No.: US 2026/0363623 A1
(43) Pub. Date: Nov. 19, 2026

(54) COUCHTOP COMPRISING A COUCHTOP EXTENSION AND A COUCHTOP BASE STATION

(71) Applicant: ORFIT INDUSTRIES, Wijnegem (BE)

(72) Inventors: Steven CUYPERS, Wijnegem (BE); Emilie CUYPERS, Wijnegem (BE); Konstantinos GIANNADAKIS, Wijnegem (BE)

(21) Appl. No.: 19/121,998

(22) PCT Filed: Oct. 23, 2023
(86) PCT No.: PCT/EP2023/079488

§ 371 Date: Apr. 17, 2025

(30) Foreign Application Priority Data
Oct. 21, 2022 (BE) 2022/5851

(51) Int. Cl. A61B 5/00

(52) U.S. Cl. CPC A61B 5/704

(57) ABSTRACT

A couchtop comprising a couchtop extension and a couchtop base station wherein the couchtop extension is releasably coupled to the couchtop base station and wherein the couchtop extension is configured to support a patient during medical diagnosis or treatment is disclosed. The couchtop extension comprises a panel extending between a first longitudinal end and a second longitudinal end along a length direction and between a first side end and a second side end along a width direction, wherein the second end of the panel comprises coupling means for releasably coupling the couchtop extension panel to the couchtop base station, wherein the couchtop base station comprises a load determination unit arranged to determine the loading of the couchtop extension.

### TECHNICAL FIELD

The present invention relates to a couchtop comprising a couchtop base station and a couchtop extension, the latter being configured to be releasably coupled to the couchtop base station. The couchtop extension is configured to support, i.e. hold, a patient during medical diagnosis or treatment. In embodiments, the couchtop extension is arranged to support a patient in a magnetic field or in a radiation zone during medical diagnosis or treatment, in particular by holding the couchtop extension, on which lies the patient, in said magnetic field or in said radiation zone. The present invention furthermore relates to the use of the couchtop.

### BACKGROUND OF THE INVENTION

Couchtop extensions and couchtop base stations are known from the prior art, such as from patent publication EP3823578 wherein the couchtop extension and the couchtop base station are respectively referred to as the extension panel and the patient support panel with pedestal.

It has been found by the present inventors that once a couchtop extension leaves the factory, it is practically impossible to know how these devices are being used during their service life. The radiotherapy clinics are busy environments. Hence, couchtop extensions are being used constantly, and it is not uncommon that improper use of these devices can occur. Improper use in particular relates to overloading of couchtop extensions (e.g. loading the couchtop extension with a higher weight than it was designed for) and to improper loading of the couchtop extensions (e.g. singular point loading on the couchtop extensions, for example because of patients supporting their entire body weight with their knees). The improper use leads to unforeseen damage. Damage sources can be accumulative viscoplastic deformations of the couchtop extensions and, in extreme cases, interlaminar and intralaminar micro-cracking inducing permanent damage.

The inventors have found that it is important to be aware of such damage sources to the couchtop extension in order to prevent performance degradation, by performing timely maintenance on the couchtop extensions: for example by means of well-timed preventive maintenances, or overall product replacement. In medical radiation and magnetic imaging applications, an accurate positioning of the patient is after all of utmost importance. At present, there are no detailed techniques to accurately register improper use events and couple them to the mechanical and functional performance when a field technician performs couchtop extensions servicing such as couchtop maintenance.

It is furthermore crucial to minimize the amount of foreign material in the couchtop extension, as this foreign material tends to deteriorate the quality of the medical treatment or diagnosis because it affects the homogenous character of the couchtop extension.

### DESCRIPTION OF THE INVENTION

It is the aim of the present invention to provide a couchtop which enables better maintenance by taking into account knowledge regarding the improper use of the couchtop extension. Simultaneously the couchtop should ensure that the medical treatment or diagnosis is disturbed as little as possible, in particular by providing a couchtop extension with a substantially homogenous character.

To that end, the present invention provides a couchtop according to the first claim. As stated before, the couchtop comprises a couchtop base station, for example mounted on a pedestal or a robotic arm, and a couchtop extension configured to be releasably coupled to a couchtop base station and configured to support a patient during medical diagnosis or treatment. Couchtop base stations in medical facilities are typically found in treatment/diagnosis rooms. They are for example mounted on robotic arms which correctly position the patient for a treatment or a diagnosis. In an embodiment, the couchtop extension is arranged to support the patient in a magnetic field or in a radiation zone during medical diagnosis or treatment, in particular by holding the couchtop extension, on which lies the patient, in said magnetic field or in said radiation zone. The medical diagnosis could for example be the medical imaging prior to a medical treatment. The magnetic field is for example present in case the medical diagnosis comprises an MRI machine. The radiation zone is for example a zone in which a patient is placed when he/she is being irradiated by means of radiation, such as X-ray radiation, gamma radiation, proton radiation or alpha particle radiation for the purposes of medical diagnosis or treatment. The couchtop extension comprises a panel extending between a first longitudinal end and a second longitudinal end along a length direction. The first longitudinal end and second longitudinal end are also referred to as respectively the cranial end and the caudal end of the panel. The panel further extends between a first side end and a second side end along a width direction. The couchtop comprises coupling means for releasably coupling the couchtop extension panel to the couchtop base station. Such a couchtop extension and couchtop base station are for example known from patent publication EP3823578, which is therefore incorporated herein by reference. According to the present invention, the couchtop base station comprises a load determination unit arranged to determine the loading of the couchtop extension. The load determination unit determines loading information indicative of the loading of the couchtop extension. The “loading information” preferably means the quantitative loading level. In an embodiment, as will be described further below, the “loading information” additionally means the qualitative type of loading such as torsion loading around an axis extending in the length direction or bending loading around an axis in the width direction. By providing the load determination unit in the couchtop base station, the loading of the couchtop extension can be determined without providing much foreign material in the couchtop extension, in particular in the panel of the couchtop extension. The loading information can be used to determine improper use of the couchtop extension for example when the measured loading level exceeds a predetermined threshold level stored in a data storage medium. The loading information could for example be a level of bending load applied on the coupling means. The surpassing of a predetermined threshold level bending load could for example demonstrate that the couchtop extension has been improperly used to support a patient whose weight exceeds the weight threshold for the couchtop extension in question. The surpassing of a predetermined threshold level bending load could for example also demonstrate that the couchtop extension has been improperly used to support a lot of weight on a limited area of the couchtop extension (for example due to a patient standing on the first longitudinal end of the couchtop extension instead of lying down on the couchtop extension). In both cases, the bending load at the coupling means of the couchtop will after all be high, and the load determination unit will detect said high bending load. The user can for example use the loading information, e.g. the level of bending load, to plan preventive maintenance to the couchtop extension. In an embodiment, the couchtop base station comprises a data storage medium for storing the loading information directly in the couchtop base station. The couchtop could also comprise a processor for pre-processing the loading information, for example by applying data reduction on the loading information. The user could then for example directly access the loading information or the pre-processed loading information from the data storage medium in the couchtop base station. Alternatively, the data storage medium could be external to the base station, and the base station could transmit the loading information, or a pre-processed version of the loading information, to the external data storage medium. The external data storage medium could for example be a cloud-based data hosting service. The user could receive said loading information or said pre-processed loading information by directly consulting the external data storage medium. In an embodiment, it is not the user of the couchtop extension that directly consults the external data storage medium, but it is an external company that directly consults the external data storage. The external company is for example the manufacturer of the couchtop extensions or a contractually appointed maintenance firm. The external company can subsequently inform the user of the couchtop extension of the improper use, for example by planning a preventive maintenance.

According to an embodiment of the present invention, the load determination unit comprises at least one load sensor. The load sensor is preferably arranged to determine the loading of the couchtop extension by measuring the load exerted on the couchtop base station by the couchtop extension when the couchtop extension is coupled to the couchtop base station. Indeed, the weight of the patient on the couchtop extension loads the couchtop extension, which in turn loads the couchtop base station. By determining the loading on the couchtop base station, the load determination unit can determine the loading of the couchtop extension, and thus the weight and/or weight distribution of the patient. This allows to monitor improper use of the couchtop extension, for example by determining the bending load at the couchtop base station as explained above. According to an embodiment of the present invention, the load sensor is a strain gauge arranged to measure the amount of straining of the couchtop base station due to the load exerted on the couchtop base station by the couchtop extension when coupled to the couchtop base station. By measuring the amount of strain of the couchtop base station, one can determine the bending load at the couchtop base station, and thus the loading of the couchtop extension. This enables to monitor improper use of the couchtop extension.

According to an embodiment of the present invention, the coupling means comprise a part provided on the couchtop extension and a part provided on the couchtop base station. Preferably, the part of the coupling means which is provided on the couchtop extension, is provided on the second longitudinal end of the panel. Preferably, the part of the coupling means which is provided on the couchtop base station comprises one of a) a tab delimited by an upper surface and a lower surface, and b) a cavity delimited by an upper surface and a lower surface. The part of the coupling means which is provided on the couchtop extension comprises the other one of the tab and the cavity. The cavity is configured to receive the tab upon coupling the couchtop extension to the couchtop base station such as to support the couchtop extension with the couchtop base station. In an example, the part of the coupling means which is provided on the couchtop base station comprises the tab and the part of the coupling means which is provided on the couchtop extension comprises the cavity. According to an embodiment of the present invention the load sensor is provided on the part of the coupling means which is provided on the couchtop base station. In the preceding example the load sensor is for example provided on the tab of the couchtop base station. According to an embodiment of the present invention the load sensor is provided on the upper surface or lower surface of the tab or cavity of the part of the coupling means which is provided on the couchtop base station. In the preceding example the load sensor is for example provided on the upper surface or lower surface of the tab of the couchtop base station. According to an embodiment of the present invention the upper or lower surface of the tab or cavity of the part of the coupling means which is provided on the couchtop base station on which a load sensor is provided, is provided with a recess wherein the load sensor is received. Providing the load sensor in a recess protects the load sensor from the surroundings.

According to an embodiment of the present invention, the panel is made of a radiolucent material such that the interference of the medical radiation by the panel is reduced. The part of the coupling means which is provided on the couchtop extension could for example be made of a non-radiolucent material.

According to an embodiment of the present invention, the tab or cavity of the part of the coupling means which is provided on the couchtop base station comprises multiple load sensors. Preferably at least one load sensor is provided on the upper surface and at least one load sensor is provided on the lower surface of the tab or cavity of the part of the coupling means which is provided on the couchtop base station. This allows to more accurately determine the loading information.

According to an embodiment of the present invention, the part of the coupling means comprising the tab, further referred to as the first tab, comprises a further tab, referred to as the second tab. The first tab and the second tab are respectively proximate to the first side end and the second side end of the panel, in particular when the couchtop extension and the couchtop base station are coupled. Accordingly, the part of the coupling means comprising the cavity, further referred to as the first cavity, comprises a further cavity, referred to as the second cavity. The first cavity and second cavity are respectively proximate to the first side end and the second side end of the panel, in particular when the couchtop extension and the couchtop base station are coupled. The set of cavities are configured to receive the set of tabs upon coupling the couchtop extension to the couchtop base station such as to support the couchtop extension with the couchtop base station. The embodiments described above with respect to the integration of the load sensor on the tab or cavity applies to one of the tabs or one of the cavities of the part of the coupling means which is provided on the couchtop base station. Alternatively, at least one load sensor is provided on each cavity or tab in the part of the coupling means which is provided on the couchtop base station. The embodiments described above with respect to the integration of the load sensor on the tab or cavity then applies to both tabs or cavities of the part of the coupling means which is provided on the couchtop base station. Providing load sensors on both tabs or cavities allows to determine torsion exerted around an axis in the length direction. The load determination unit comprising the load sensors on both tabs or cavities thus enables to determine both quantitative and qualitative loading information. In a preferred embodiment, each of the two tabs or cavities in the part of the coupling means which is provided on the base station, comprises a load sensor on the upper surface and a load sensor on the lower surface. In an alternative embodiment, each of the two tabs or cavities in the part of the coupling means which is provided on the base station, comprises a load sensor on the upper surface, and not on the lower surface. In an alternative embodiment, each of the two tabs or cavities of the part of the coupling means which is provided on the base station, comprises a load sensor on the lower surface, and not on the upper surface.

According to an embodiment of the present invention, the coupling means further comprise identification means enabling the couchtop base station to identify the couchtop extension connected to it. Preferably, the identification means enable the load determination unit to take into account the type of couchtop extension when determining the loading of the couchtop extension. Different types of couchtop extensions for example have different weight limits. The couchtop base station could for example be configured to pre-process the loading information by selecting “overloading events” for the couchtop extension in question. According to an embodiment of the present invention, the loading information comprises a counter indicating the number of times the couchtop extension has been connected to the couchtop base station. Preferably, the identification means enable the couchtop base station to identify the couchtop extension connected to the base station such that the couchtop base station can update the counter by one for the identified couchtop extension. The number of times a couchtop extension has been connected is valuable information to take into account when determining the servicing of the couchtop extension. According to an embodiment of the present invention, the loading information comprises a counter indicating the number of times the couchtop extension has been used to support a patient. Preferably, the identification means enable the couchtop base station to identify the couchtop extension connected to the base station such that the couchtop base station can update the counter by one for the identified couchtop extension upon the load determination unit determining that the couchtop extension has been loaded. The number of times a couchtop extension has been used is valuable information to take into account when performing the maintenance of the couchtop extension.

According to an embodiment of the present invention, the couchtop base station comprises a communication module arranged to send the determined couchtop extension loading information to the external data storage medium such as to a cloud-based hosting service. As described above, the communication module can also send pre-processed loading information to the external data storage medium.

It is a further object of the present invention to provide for a use of the couchtop as described above. Preferably the use comprises the couchtop extension being coupled to the couchtop base station, and determining the loading of the couchtop extension by means of the load determination unit. Preferably, the use comprises supporting the patient in a magnetic field or in a radiation zone as described above.

### FIGURES

FIG. 1 shows a perspective view of a couchtop according to an embodiment of the present invention.

FIG. 2 shows a cross-sectional view through the couchtop of FIG. 1 across the first tab and first cavity of the coupling means.

### BRIEF DESCRIPTION OF THE FIGURES

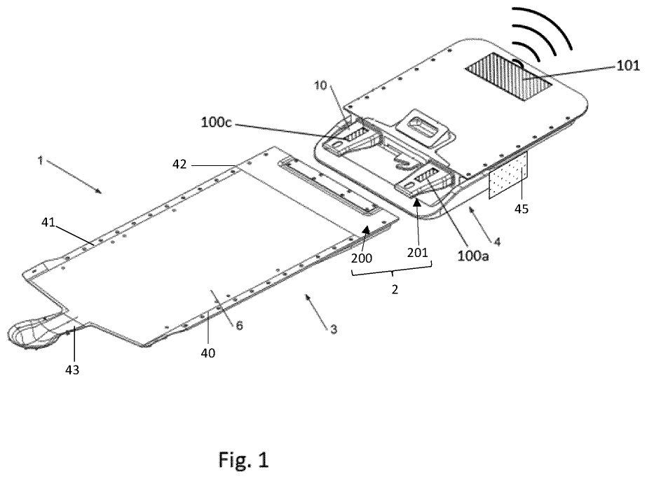

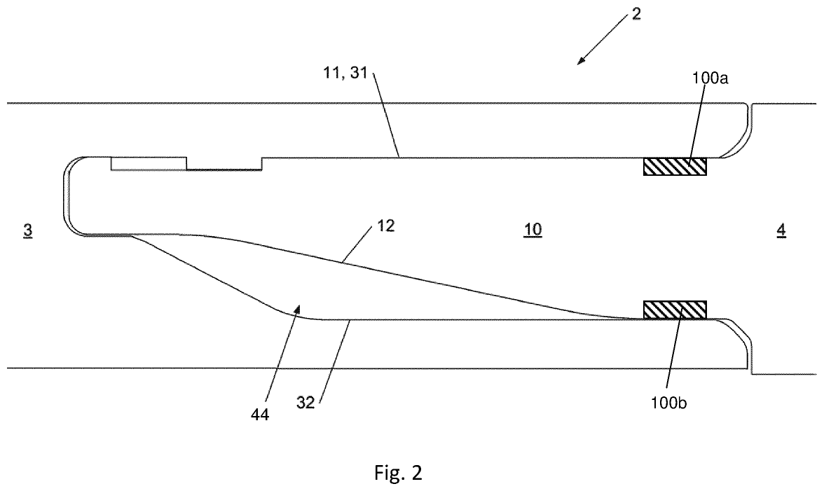

As shown in FIG. 1, the couchtop 1 comprises a couchtop base station 4 mounted on a pedestal or a robotic arm (not shown), and a couchtop extension 3 configured to be releasably coupled to a couchtop base station. The couchtop is arranged to support a patient in a magnetic field or in a radiation zone during medical diagnosis or treatment, by holding the couchtop extension, on which lies the patient, in said magnetic field or in said radiation zone. The couchtop extension 3 comprises a panel 6 extending between a first longitudinal end 43 and a second longitudinal end 42 along a length direction and between a first side end 40 and a second side end 41 along a width direction. The couchtop comprises coupling means 2 for releasably coupling the couchtop extension panel to the couchtop base station. Such a couchtop extension and couchtop base station are for example known from patent publication EP3823578, which is therefore incorporated herein by reference. The distinguishing features are a communication module 101 and a load determination unit provided in the couchtop base station. The load determination module is arranged to determine loading information indicative of the loading of the couchtop extension. The loading information can be used to determine improper use of the couchtop extension for example when the measured loading level exceeds a predetermined threshold level stored in a data storage medium. The loading information could for example be a level of bending load applied on the coupling means. The weight of the patient creates a profile of bending loads along the couchtop extension. The bending load is typically highest at the coupling means, i.e. at the interface between the couchtop extension and the couchtop base station. The surpassing of a predetermined threshold level bending load could for example demonstrate that the couchtop extension has been improperly used to support a patient whose weight exceeds the weight threshold for the couchtop extension in question or that it has been improperly used to support a lot of weight on a limited area of the couchtop extension (for example due to a patient standing on the first longitudinal end of the couchtop extension instead of lying down on the couchtop extension). In both cases, the bending load at the coupling means of the couchtop will after all be high, and the load determination unit will detect said high bending loads. The user can use the loading information, e.g. the level of bending load, to plan preventive maintenance to the couchtop extension. The communication module 101 is in communication with a data storage medium which is external to the couchtop base station. The couchtop base station is arranged to transmit the loading information to the external data storage medium by means of the communication module. The external data storage medium could for example be a cloud-based storage service. The user could receive said loading information by consulting the external data storage medium. Alternatively, or additionally, an external company could consult the external data storage medium. Furthermore, the couchtop base station comprises a display 45, on which an overloading incident can be displayed to the user of the couchtop extension. The load determination unit comprises four load sensors implemented as strain gauges (three load sensors are explicitly shown in the figures: 100a, 100c in FIG. 1 and 100a, 100b in FIG. 2). By measuring the strain of the couchtop base station, one can determine the loading of the couchtop extension. This enables to monitor improper use of the couchtop extension. The coupling means 2 comprise a part 200 provided on the couchtop extension 3 and a part 201 provided on the couchtop base station 4. The part 201 of the coupling means on the couchtop base station comprises two tabs 10 each delimited by an upper surface 11 and a lower surface 12. The part 200 of the coupling means on the couchtop extension comprises two cavities 44 each delimited by an upper surface 31 and a lower surface 32. The cavities are configured to receive the tabs upon coupling the couchtop extension to the couchtop base station such as to support the couchtop extension with the couchtop base station. One load sensor is provided on the upper surface and one load sensor is provided on the lower surface of each of the two tabs 10 (only three are explicitly shown). Each load sensor is provided in a dedicated recess. By proving the four load sensors as described above, the “loading information” comprises the quantitative loading level, as well as the qualitative type of loading such as torsion loading around an axis extending in the length direction or bending loading around an axis extending in the width direction. The qualitative type of loading could for example be a pure bending around an axis extending in the width direction. This results in a similar measurement by the load sensors on each of the two tabs. The load sensors on the upper surface of the tabs will register an extending normal stress, whilst the load sensors on the lower surface of the tabs will register a compressive normal stress. However, if the patient weight is not evenly distributed along the width direction, then the couchtop extension will torsion around an axis extending in the length direction.

1. A couchtop comprising a couchtop extension and a couchtop base station wherein the couchtop extension is releasably coupled to the couchtop base station and wherein the couchtop extension is configured to support a patient during medical diagnosis or treatment, the couchtop extension comprising a panel extending between a first longitudinal end and a second longitudinal end along a length direction and between a first side end and a second side end along a width direction, wherein the couchtop comprises coupling means for releasably coupling the couchtop extension panel to the couchtop base station, wherein the couchtop base station comprises a load determination unit arranged to determine the loading of the couchtop extension.

2. The couchtop according to claim 1, wherein the couchtop extension is arranged for supporting the patient in a magnetic field or in a radiation zone during medical diagnosis or treatment.

3. The couchtop according to claim 1, wherein the load determination unit comprises at least one load sensor arranged to determine the loading of the couchtop extension by measuring the load exerted on the couchtop base station by the couchtop extension when the couchtop extension is coupled to the couchtop base station.

4. The couchtop according to claim 3, wherein the load sensor is a strain gauge arranged to measure an amount of straining of the couchtop base station due to the load exerted on the couchtop base station by the couchtop extension when coupled to the couchtop base station.

5. The couchtop according to claim 3, wherein the coupling means comprise a part provided on the couchtop extension and a part provided on the couchtop base station, wherein the part of the coupling means which is provided on the couchtop base station comprises one of a tab delimited by an upper surface and a lower surface, and a cavity delimited by an upper surface and a lower surface, and wherein the part of the coupling means which is provided on the couchtop extension comprises the other one of the tab and the cavity, wherein the cavity is configured to receive the tab upon coupling the couchtop extension to the couchtop base station such as to support the couchtop extension with the couchtop base station, and wherein the load sensor is provided on the part of the coupling means which is provided on the base station.

6. The couchtop according to claim 5, wherein the part of the coupling means which is provided on the couchtop extension, is provided on the second longitudinal end of the panel.

7. The couchtop according to claim 5, wherein the load sensor is provided on the upper surface or lower surface of the tab or cavity of the part of the coupling means which is provided on the couchtop base station.

8. The couchtop according to claim 7 wherein the upper or lower surface of the tab or cavity of the part of the coupling means which is provided on the couchtop base station on which a load sensor is provided, is provided with a recess wherein the load sensor is received.

9. The couchtop according to claim 5, wherein the tab or cavity in the part of the coupling means which is provided on the couchtop base station comprises multiple load sensors, wherein at least one load sensor is provided on the upper surface and at least one load sensor is provided on the lower surface of the tab or cavity of the part of the coupling means which is provided on the couchtop base station.

10. The couchtop according to claim 5, wherein the part of the coupling means comprising the tab, further referred to as the first tab, comprises a further tab, referred to as the second tab, wherein the first tab and the second tab are respectively proximate to the first side end and the second side end of the panel, wherein the part of the coupling means comprising the cavity, further referred to as the first cavity, comprises a further cavity, referred to as the second cavity, wherein the first cavity and second cavity are respectively proximate to the first side end and the second side end of the panel, wherein the set of cavities are configured to receive the set of tabs upon coupling the couchtop extension to the couchtop base station such as to support the couchtop extension with the couchtop base station, and wherein at least one load sensor is provided on each cavity or tab in the part of the coupling means which is provided on the couchtop base station.

11. The couchtop according to claim 1, wherein the coupling means further comprise identification means enabling the couchtop base station to identify the couchtop extension connected to it.

12. The couchtop according to claim 11. wherein the identification means enable the load determination unit to take into account the type of couchtop extension when determining the loading of the couchtop extension.

13. The couchtop according to claim 11, wherein the loading information comprises a counter indicating the number of times the couchtop extension has been connected to the couchtop base station, and wherein the identification means enable the couchtop base station to identify the couchtop extension connected to the base station such that the couchtop base station can update the counter by one for the identified couchtop extension.

14. The couchtop according to claim 1, wherein the couchtop base station comprises a communication module arranged to send the determined couchtop extension loading information to an external data storage medium such as a cloud-based hosting service.

15. Use of the couchtop according to claim 1, wherein the couchtop extension is coupled to the couchtop base station, and wherein the loading of the couchtop extension is determined by means of the load determination unit.
